(19) United States
(12) Patent Application Publication
(10) Pub. No.: US 2026/0160686 A1
(43) Pub. Date: Jun. 11, 2026

(54) INDICATOR LABEL FOR OPTICALLY ASCERTAINING THE CONCENTRATION OF AN ANALYTE IN A FLUID MIXTURE

(71) Applicant: Alexander WANNENWETSCH, Willmars (DE)

(72) Inventor: Alexander Wannenwetsch, Willmars (DE)

(73) Assignee: Alexander Wannenwetsch, Willmars (DE)

(21) Appl. No.: 19/110,925

(22) PCT Filed: Nov. 17, 2023
(86) PCT No.: PCT/DE2023/100889

§ 371 Date: Mar. 12, 2025

(30) Foreign Application Priority Data
Nov. 25, 2022 (DE) 10 2022 131 238.0

(51) Int. Cl. G01N 21/64; G01N 33/00; G01N 33/02

(52) U.S. Cl. CPC G01N 21/643; G01N 33/0081; G01N 33/02; G01N 2021/6439

(57) ABSTRACT

A package includes an interior filled with a modified atmosphere and an indicator label. The indicator label includes a carrier layer, an adhesive layer applied flatly on the an underside of the carrier layer, and an indicator substance dot on the an upper side of the carrier layer, which is opposite to the underside. The indicator substance dot includes an indicator substance that enables a determination of a presence and/or a concentration of an analyte in a multicomponent fluid mixture using an optical sensor through an excitation of the indicator substance with excitation light and reading fluorescence light emitted by the indicator substance using the optical sensor. The indicator substance dot has a uniform thickness in a direction that is perpendicular to the upper side of the carrier layer, and the indicator label is adhered to a location of the packaging that is sufficiently transparent for excitation and fluorescent light.

The present invention relates to a label with an indicator substance for optical determination of an analyte, in particular for determining the concentration of the analyte, in a multicomponent fluid mixture, in particular gases, the label comprising a carrier layer, an adhesive layer applied flatly on the underside of the carrier layer, and an indicator substance dot on the side of the carrier layer opposite the adhesive layer, wherein the indicator substance dot contains an indicator substance that allows for determining the presence and, in particular, the concentration of the analyte using optical means. Furthermore, the present invention relates to a tape with a plurality of such labels arranged consecutively as well as methods for manufacturing such labels or label tapes. Additionally, the invention relates to a packaging that comprises at least one of the inventive labels and to a packaging machine for packaging food or other sensitive goods in such a packaging.

For food and other sensitive goods, proper packaging is essential to ensure the longest possible shelf life. The packaging should, after sealing, be at least germ- and dirt-proof. To further extend shelf life, gas-tight packaging is additionally used, where, assuming proper sealing, almost no gas exchange occurs between the atmosphere inside the packaging and the external atmosphere. For this purpose, packaging made from pore-free materials is employed, which, ideally, allow only minimal diffusion-based gas exchange to occur.

Preferred are, in particular, packagings made of plastic film, such as in the form of a pouch, or two-part packaging consisting of a bottom tray (which can be made of plastic or coated cardboard) and a sealing film, such as a transparent plastic film. To further reduce particle diffusion through the packaging, a metal foil or metal-coated plastic film can also be used. Such gas-tight packaging allows for creating a modified atmosphere within the sealed packaging, which is why these are also referred to as MAP (Modified Atmosphere Packaging) industrial packaging.

This modified atmosphere typically consists of one or more inert gases. Nitrogen is predominantly used for economic reasons. Inert gases are employed to prevent oxidation of the food or other goods by reactive atmospheric oxygen. The goal of a typical MAP packaging is therefore to ensure that the residual oxygen level within the package is as low as possible after sealing.

This modified atmosphere is intended to maintain the sealed packaging for as long as possible. The weak point of the packaging is therefore less the packaging material itself—even if it has a certain diffusion-based permeability—but rather the sealing. During the sealing process, typically performed by machines, incomplete sealing can occur for various reasons. For one, the packaging may not be properly aligned during sealing, so that the sealing stamp only seals some areas partially or not at all. Additionally, particles of the packaged goods can get between the sealing parts of the packaging, compromising the seal.

To ensure that the produced and delivered food items can actually last until the printed “best before” date, it is therefore common to test the seal integrity of the packaging immediately after sealing. This is done by exposing the packaging to mechanical forces or varying pressure, which will cause a rapid change in the modified atmosphere inside the packaging if the seal is faulty. In a subsequent step, the composition of the atmosphere is then measured, and if it deviates from the expected or previously measured value, the packaging is discarded.

The examination of the atmosphere inside the packaging can be performed destructively, for example, by puncturing the packaging with a measuring probe or lance to take a sample of the atmosphere. However, non-destructive methods are also known in the state of the art.

In methods where the composition of the atmosphere—especially the residual oxygen—is determined using optical means, a fluorescent dye is located inside the packaging, for example, printed on the inner side of the packaging film. This dye is excited from the outside using light at the appropriate frequency, and its emission response is measured over time. From these measured fluorescence parameters—specifically the emission intensity and/or the decay time after the excitation is turned off—the oxygen concentration can be inferred, provided the system has been calibrated beforehand.

With the porphyrin dyes typically used, it is particularly notable that, in the presence of oxygen, the decay time shortens, and the intensity of the emission response is reduced. This phenomenon is referred to as “oxygen-induced quenching of fluorescence.” Such a method is described, for example, in the international publication WO 2018/202784 A1.

In another international patent application publication, WO 2017/125386 A2, a pouch packaging machine is presented. This machine has means to print sensor dots on transparent areas of the packaging, which can then optically determine the residual oxygen content in the prescribed manner after the pouch has been sealed. Because measurements are taken on the moving pouches as they pass by the sensor, the document proposes that the sensor dots be designed to be elongated, with their longitudinal direction oriented along the movement direction of the pouches. This design gives the optical sensor more time to correctly detect the sensor dots.

In the international publication WO 2018/011307 A1, a packaging machine and a method for packaging food in two-part packaging made of grid-like arranged shell-shaped bottom trays, which are closed with a sealed transparent film, are presented. In this case, a sensor dot containing a fluorescent dye is printed on the underside of the film on the inside of each packaging. An optical sensor of the packaging machine determines the residual oxygen content of the package after sealing and, if necessary, a leak test. Based on the measured value, an individual “best before” date is calculated for each package and stored in an RFID label affixed to the outside of the packaging.

The published European patent application EP 0449798 A2 presents a method for quality control of packaged organic materials, in which a planar optical sensor element is inserted into the packaging. The sensor element is in contact with the food and can detect changes in the gas composition within the interior of the packaging. This is achieved because the sensor element contains chemical substances sensitive to various analytes. These analytes are degradation products of the packaged food, and their concentration changes can be optically detected. The sensor element consists of an indicator layer, which is covered in the direction of the food by a hydrophobic polymer layer. The side opposite the polymer layer is attached to the inside of the packaging. The disadvantage of the proposed sensor element is its relatively large spatial extent, as well as the fact that no measures are taken to design the indicator layer in a way that ensures a reliable measurement signal. On the other hand, the sensor element is relatively bulky and has a large surface area, thereby reducing the space available in the interior of the packaging for the actual packaged goods. Furthermore, the resource consumption caused by such a large sensor element is not very advantageous when used on a large scale. For instance, in Germany alone, hundreds of thousands of MAP-packaging units are produced daily.

The publication WO 2022/055433 A1 introduces an oxygen sensor and an oxygen sensor film label containing the sensor. The oxygen sensor comprises a multilayer polyelectrolyte film and a dye carrier with a fluorescent dye. The oxygen sensor label includes such an oxygen sensor, which is additionally covered by a food layer and an overlying protective layer. Furthermore, the underside of the oxygen sensor or the oxygen sensor layer includes an adhesive layer designed to attach the sensor label to the surface of a packaging. The disadvantage of this label lies in its relatively complex structure and, as a result, high production costs. Additionally, it has a relatively large surface area, which means that to ensure correct optical reading, a correspondingly large transparent area must be present within the packaging. On the other hand, the presence of such a large and thick label within the packaging's interior can also be disruptive or bothersome for the consumer.

A better solution for simply and economically providing a means to optically determine the atmosphere inside a package in a non-destructive manner is presented in the published US patent application US 2013/0177480A1. This document proposes a freely positionable pressure measuring probe which can be measured optically from the outside and which comprises a solid composition containing a fluorescent dye on a carrier layer, wherein an adhesive layer is provided on the underside of the carrier layer for attaching the pressure measuring probe to the inside of a package. It is proposed to provide these comparatively small pressure measuring probes, which can be produced from cheap resources and which also allow, in particular, the optical determination of the oxygen concentration of the packaging atmosphere, arranged in large numbers one behind the other on space-saving rolled-up tapes.

The disadvantage of this solution, however, is that no measures are provided to ensure sufficient accuracy of the fluorescence measurement. In particular, the optical measurement of the sealed package is usually carried out on the packages moving past stationary sensors. In order to obtain a uniform signal, it is therefore important that the dye density in the sensor's field of view does not change during the measurement time. With the point-shaped probe labels proposed by US 2013/0177480A1, it is hardly possible to measure moving packaging, as the layer containing the fluorescent dye has too small an area on the one hand and a uniform apparent dye density is not ensured on the other.

Against this background, it is the object of the present invention to provide a means for the failure-free determination of an analyte, in particular determination of its concentration, in a package which can be produced quickly and cheaply and which permits accurate measurement even in the case of relative movement between the label and the optical sensor. Furthermore, the packaging provided with the means should, if possible, be able to be graphically designed and, in particular, printed freely and without having to take into account the technical means provided for enabling the determination of the concentration.

This object is solved in a first aspect of the invention by an indicator label according to claim 1, which can be produced according to the method of claim 10, and a tape with a plurality, in particular a multiplicity, of such labels lined up one behind the other according to claim 12, which can be produced according to the method presented in claim 13. In a further aspect, the present invention proposes a package with at least one such indicator label on the inner side facing the, optionally modified, atmosphere according to claim 14. In a third aspect, the invention relates to a packaging machine according to claim 15 and to a method according to claim 20 for packaging foodstuffs or other sensitive goods in a package with an indicator label according to the invention.

The first aspect of the present invention is an indicator label according to one of claims 1 to 9. This label comprises, in a generally known manner, a support or carrier layer with an adhesive layer applied in a planar fashion on its underside, with the carrier layer carrying an indicator substance dot on its upper side opposite the underside. The indicator substance dot contains an indicator substance whose properties change depending on the presence, and in particular the concentration, of the analyte to be detected in a manner that can be detected optically from the outside.

In particular, the indicator substance may be a luminescent dye—specifically a fluorescent or phosphorescent dye—that emits light of a different wavelength when excited with light of a certain wavelength. The determination of the analyte's concentration is possible with these luminescent-based dyes because the luminescence parameters are dependent on the presence and generally also on the concentration of the analyte.

For example, in the case of porphyrin dyes, the presence of oxygen reduces both the decay time and the intensity of the fluorescence response to optical excitation. In other words, in the presence of oxygen, the emitted fluorescence light from the dye is less intense, and the fluorescence signal decays faster after the excitation light is turned off than in the absence of oxygen. These changes become more pronounced as the concentration of oxygen increases. Thus, by measuring the intensity and/or decay time of the fluorescence response signal, the concentration of oxygen in the vicinity of the dye can be determined.

Characteristic of the indicator label according to the present invention is the uniform thickness of the indicator substance dot, which in some embodiments is also formed as an indicator (substance) layer covering the entire surface of the label or as an elongated indicator (substance) strip across its area. The deviation in the relative thickness of the indicator substance dot varies between two points, at least in a longitudinal direction parallel to the top side of the support layer, preferably by no more than 10 percent, and particularly preferably by no more than 1 percent. This ensures that during optical measurement of the indicator label, the visible surface density of the indicator substance detectable by the optical sensor remains constant as the label moves through the sensor's detection range. This ensures that a uniform optical signal can be detected and that the accuracy of the analyte determination is not negatively influenced by relative movement between the sensor and the label.

The thickness of the indicator layer does not necessarily need to be uniform in two perpendicular directions. Rather, it is sufficient if the indicator substance dot has uniform thickness in one direction only—meaning the indicator substance dot has strip-like areas of uniform thickness. Variations in thickness perpendicular to this direction can be tolerated, provided that during measurement, any relative movement between the sensor and the indicator label only occurs parallel to the direction of uniform thickness.

In preferred embodiments, the indicator labels according to the invention advantageously have relatively small dimensions of just a few millimeters and are provided in particularly preferred embodiments in large numbers, sequentially on continuous strips of material wound into rolls.

The indicator labels are produced according to the method of the present invention by printing the indicator substance dot onto a label blank or label substrate. For this purpose, a label blank is first provided, which comprises a carrier layer and an adhesive layer on its underside. Practically speaking, the side of the adhesive layer opposite to the carrier layer should be covered in a known manner with a detachable coating that adheres only lightly to the adhesive layer, in order to avoid unwanted sticking of the label.

The label blank can already match the shape of the later final label in its outline. However, it is preferred to provide a blank that comprises a label section and a surplus section that is adjacent to the label section or surrounds it. The indicator substance dot is printed onto this blank such that it extends beyond at least one, preferably two, especially opposite edge areas of the label section. A more or less uniform distribution of the indicator substance over the area of the indicator substance dot is achieved by the self-leveling properties of the ink used.

The portion of the indicator substance that extends onto the surplus section will be removed together with the surplus section itself after the drying of the printed indicator substance. The result is an indicator label according to the invention with an indicator substance dot that has a uniform thickness at least in a direction parallel to the surface of the carrier layer.

As an example, a label blank can be used where the label section has a round or oval shape and is surrounded by a surplus section on all sides. Such a blank can, for example, be manufactured by forming a label section of a desired shape from a blank that comprises a support layer with an adhesive layer on the underside, preferably with a detachable coating beneath it. Care must be taken during this process to avoid damaging the detachable coating, so that the label section and surplus section remain spatially connected. Various indicator substances can now be printed onto such a blank.

In some embodiments of the invention, an indicator substance dot is printed that fully covers the label section and extends beyond all sides onto the surplus section. During the drying process, the solid particles dispersed in the liquid indicator substance tend to accumulate at the edge of the printed point, resulting in the greatest thickness there after drying. Conversely, the center of the printed point has the least amount of indicator substance after drying. However, the gradient in thickness is relatively low, for example, ten or more percent of the extent of the indicator substance dot in this direction. This ensures that after the removal, particularly the detachment, of the surplus section surrounding the label section, the remaining central portion of the indicator substance dot on the label section retains a largely uniform thickness. This uniform thickness is also present in the direction perpendicular to the surface of the label in these embodiments.

In alternative embodiments, the division of the support layer into a label section and surplus section(s) occurs only after printing. This has the advantage of allowing a cleaner separation between the part of the indicator substance dot on the label portion and the (thicker) portions of the indicator substance dot on the surplus section.

The disadvantage of the previously described embodiments of the inventive labels lies in the fact that to detect a label passing a sensor at a relatively high speed, a certain spatial extension of the label is necessary. For example, if the label moves past the sensor at a speed of one meter per second and ten data points are required for reliable optical measurement and the sensor records data points every millisecond, the label would have to have a length in the direction of movement of at least ten millimeters.

For a uniformly coated indicator substance label, according to the previously described embodiments, which has a round shape, this would mean a surface area of approximately 80 mm² would need to be covered with the indicator substance. However, the fluorescence dyes used for oxygen detection are comparatively expensive, with prices of several thousand euros per liter. Therefore, a resource-saving use of these substances is strongly preferred for economic reasons. Additionally, an extension perpendicular to the direction of motion by ten millimeters is neither necessary nor desired. The reason is that a changing width of the indicator substance dot would lead to varying measurement signals. While this could be avoided by using labels with a rectangular footprint or corrected during measurement if the shape of the indicator substance dots is known, the effort involved in this second case can be saved if the indicator substance dot itself has a shape that is at least substantially rectangular, with the longer side oriented in the direction of motion.

The shape of the label section itself is of secondary importance within the context of the present invention. The label section or the finished indicator label can be round, oval, rectangular, or have another functional shape. For reasons of easier manufacturing and handling, a round or rectangular shape—such as a square—is most practical.

On a label section or label, the indicator substance dot is preferably printed in the form of a rectangle, with the two opposite ends of the rectangle extending beyond the label section or label itself and, during the manufacturing process according to the inventive method, located on the surplus section. After the liquid indicator substance or the inks containing the indicator substances have dried, the surplus section is removed on both sides so that only a rectangular, nearly oblong strip remains on the top side of the support layer, with the exception of possibly curved short end sections.

In the longitudinal direction, this indicator substance strip has the uniform thickness desired by the invention. Perpendicular to this direction, the thickness does vary; however, this is insignificant if the label is applied in the packaging machine in such a way that the longitudinal side of the rectangular indicator substance strip is oriented parallel to the direction of motion of the packaging within the packaging machine.

According to the method of the inventions, individual indicator labels according to the invention can be produced. However, the labels are preferably manufactured on a roll, in which, in accordance with the similarly claimed manufacturing method for an indicator label roll, a blank in the form of a band made of an adhesive layer embedded between an upper-side carrier layer and a lower-side peel-off layer is provided, and in this blank, indicator label sections are separated in the desired form simultaneously or successively one after the other. This separation can be carried out by punching or by cutting, for example by laser cutting, whereby during this process care must be taken to only cut through the carrier and optionally also the adhesive layer, but not to damage the peel-off layer. To achieve this, it is advantageous to select a material for the adhesive/peel-off layer that is less easily separable by the separation method used than the carrier layer. The contiguous carrier layer area surrounding the successive indicator label sections, unless further subdivided, then forms the surplus section.

In the previously described manner, in the method of the invention, indicator substance spots are printed onto the label sections in such a way that they are located at one location, preferably at two locations, in particular two opposite locations, or in some embodiments, they protrude on all sides beyond the label section so that the edges are located on the surplus section. The indicator substance ink is left to dry after printing or actively dried, and after drying, the surplus section is removed. To simplify this in industrial production, instead of a large, continuous surplus section, it can, for example, be divided into smaller, easily machine-removable parts during the process of separating the label sections. After removing the surplus section, what remains is a roll with the inventive indicator labels arranged thereon, consisting of a carrier layer, a lower-side adhesive layer applied over the entire surface, and an indicator substance spot on the upper side of the carrier layer.

The indicator substance spot may, as described above, cover the entire upper side of the carrier layer or only part of it, for example in the form of a rectangle leaving areas of the label section uncovered on the right and left. On one, preferably two opposite sides, the indicator substance spot, however, preferably ends flush with the label or the label section.

The printing of the indicator substance spots may take place either before or after the separation of the label sections.

Another aspect of the present invention is a packaging for food or another sensitive good, for example, a packaging with a modified atmosphere, in which an indicator label according to the invention is glued at a location visible from the outside. The packaging may, for example, be a sachet with at least one transparent area or a tray-type packaging closed with a top film. The term “visible from the outside” is to be understood here in such a way that the location is sufficiently transparent for externally applied excitation light and simultaneously for internally incident fluorescence light emitted by the indicator substance in the indicator substance spot of the indicator label, which usually has a different (lower) frequency than the excitation light.

The inventive packaging may be unprinted or have a print at the visible, i.e., sufficiently transparent, location; however, this print is designed so that it is sufficiently transparent for excitation and fluorescence light. This can be achieved by using ink that is transparent to the excitation and fluorescence light for the printing.

Alternatively or additionally, the print may have everywhere, however at least in the area of the surface occupied by the indicator label on the inside of the packaging, printed and unprinted areas, whereby the unprinted areas make up at least 5%, preferably at least 10%, in particular at least 25% of the area of the indicator label. Printed and unprinted areas are preferably alternated, in particular in the form of a one-dimensional (line) or two-dimensional grid of dots. The unprinted areas each have a height and width that is greater than the wavelength of both the excitation and the (longer-wave) fluorescence light.

In particular embodiments of the packaging, the print is applied by digital printing and comprises a grid of halfltone dots with a distance between adjacent points of the grid of at least 1.05 times the radius of a grid point, in particular at least 1.1 times, for example, 1.2 to 2.0 times a grid point radius.

A third aspect of the present invention is a packaging machine for packaging food or other sensitive goods in a packaging according to the previous aspect, wherein the machine comprises labeling means for gluing an inventive indicator label onto the inner side of the packaging, which comes into contact with the atmosphere inside the packaging at a suitable, in particular transparent, location of the inventive indicator label before sealing the packaging.

Furthermore, the inventive packaging machine preferably comprises at least one optical sensor for detecting and measuring indicator labels in the sealed packages. Using this sensor, depending on the indicator label and the sensor used, at least one analyte, such as a gas component of a modified atmosphere, can be determined in the interior of the packaging after manufacturing, thus determining the quality of the atmosphere. This allows additional information to be derived, such as the expected minimum shelf life. In principle, this can also be extended to liquid-filled packages, provided suitable indicator substances and labels are used.

In some embodiments, several optical sensors are present. These can be arranged transversely to the transport direction of the packages through the machine in essentially parallel to each other in order to measure multiple lanes of packaging simultaneously without the sensor having to change its position quickly. Alternatively, or additionally, at least two optical sensors are also present in the transport direction for each packaging lane, for example, in order to measure the atmosphere of each package at two time points after sealing. To detect leaks, a vibration or pressure change path can be located between the optical sensors.

Since, in practice, packaging commonly is not fully transparent but instead transparent sections alternate with printed or otherwise covered sections, and the transparent areas suitable for gluing the inventive label generally lie at different positions for each package, it is important that the labeling means and also the optical sensor can be aligned transversely to the transverse position of the transparent sections in which the indicator labels are to be glued. This alignment can be performed manually by adjusting the labeling means and/or the optical sensor before a run with a specific packaging design. Alternatively, or additionally, the labeling means and/or the optical sensor are mounted to be transversely movable, for example, on a sled running on a transverse rail.

Even with a given configuration of the packaging, tolerances must be compensated for, within which the transparent areas may deviate from the expected position both transversely and longitudinally. Therefore, in advantageous further embodiments of the machine, optical detection means are provided which determine the exact positions of the transparent areas individually for each package. The labeling means and/or the optical sensor are then aligned transversely for each of the packages based on the position determined for that specific packaging, so that for each package, the label is glued by the respective labeling means at exactly the same position relative to the edge of the respective targeted transparent area, and the comparatively narrow detection area of the optical sensor is at correct the lateral position of the label. To achieve this, it is necessary, as an option presented for the above-described embodiments, that the labeling means and, if applicable, the optical sensor are mounted to be transversely movable.

Here, and elsewhere in this application, “transparent” does not necessarily refer to transparency in the visible spectrum but, unless expressly stated otherwise, refers to transparency of the packaging in the wavelength range(s) relevant to the optical measurement of the indicator substance.

Furthermore, the packaging machine according to this aspect of the invention preferably uses indicator label bands with sequentially arranged inventive indicator labels. These can, for example, be rolled off a conveyor (belt) arm, which at its distal end has a means for detaching the labels and applying them to a desired position on a packaging. The means for detaching can include, for example, a deflection roller with a small radius, so that when the indicator label band runs over the deflection roller on the opposite side of the peel-off layer, the label detaches on one side due to the small radius of the deflection roller and depending on the stiffness of the carrier layer. The label can then be brought into contact with the recognized and preferred location on the packaging via the detached side and, through further detachment from the label band, can ultimately be fully transferred to the packaging.

In these preferred embodiments, the tip of the conveyor belt arm can be positioned at least in one direction transverse to the direction of motion of the packaging machine to bring a label to be applied to a packaging into the area where the optical detection means has recognized the suitable or targeted transparent area. This is possible, for example, because the arm is mounted to pivot about its z-axis or yaw axis. Alternatively, or additionally, the arm can also be mounted to move in a transverse direction, for example, using a slide. Furthermore, it is conceivable that the distal end of the conveyor belt arm can be repositioned in both directions within the plane of the packaging, that is, also in the vertical direction. For this, the arm could, for example, be rotatably mounted about its y-axis or pitch axis or be movable in the vertical direction.

The inventive method for packaging food or other sensitive goods according to this aspect of the invention in packages according to the second aspect of the invention comprises the following two steps:

In a first step, an indicator label according to the first aspect of the invention is applied to the later packaging side that comes into contact with the atmosphere inside the sealed packaging at a freely selected or specified transparent area by means of the labeling means of the packaging machine. “Freely selected” here means that the labeling means can be oriented freely in relation to a reference point of the packaging, at least prior to each run with a specific packaging design, or even better, dynamically during a run, for example, to process different packaging designs within the same run with a packaging machine or to compensate for manufacturing-related tolerances in the position of the transparent areas intended for the indicator labels on the packaging.

In a second step, a measurement of the analyte, for example, a gas component, within the packaging is performed by means of the optical sensor, which is manually or automatically aligned to the freely selected area of the packaging. This allows a post-check of the atmosphere and a determination of the expected shelf-life to be achieved. Additionally, the sealing quality can also be checked if, for each packaging, a second measurement of the analyte(s), in particular gas components, takes place at a different time interval, either by a spatial movement of the optical sensor or simply by using a second optical sensor positioned further back in the transport direction.

Further advantageous embodiments are contained in the dependent claims and will be described in detail below. They also form part of the present invention in any combination with one another, provided that they are not obviously mutually exclusive.

In the indicator label according to the first aspect of the present invention, the relative thickness deviation is not more than 10%, in particular not more than 1%, at least in one direction, preferably in both directions perpendicular to the upper side of the backing layer. The preferred absolute thickness of the indicator layer or indicator layer spot is between 5 and 500 micrometers, in particular between 1 and one hundred micrometers.

The carrier layer of the indicator label according to the invention can consist of cellulose or plastic, in particular polypropylene or polyester, and preferably has a thickness of between 20 and 1000 micrometers, in particular preferably between 50 and 200 micrometers.

The adhesive layer that bonds the proper indicator label to the inside of the packaging preferably contains or consists of a food-compatible adhesive.

The indicator substance dot on the upper side of the carrier layer of an indicator label according to the invention is preferably elongated, with a length-to-width ratio of between 2 and 20, preferably between 5 and 10. In particular, the indicator substance dot is preferably rectangular in shape with a width of between 0.5 and 2 millimeters and a length of between 4 and 10 millimeters. In particularly preferred embodiments, the indicator dot is approximately one millimeter wide and approximately 7 or 8 millimeters long.

In some embodiments of the indicator label according to the invention, the indicator substance dot is flush with the edge of the carrier layer at at least one point, preferably at two, especially opposite points. This maximizes the length of the indicator substance dot or strip.

The adhesive layer preferably has a thickness of between 10 and 200 micrometers, in particular between 50 and 100 micrometers.

The inventive indicator label is in preferred embodiments essentially transparent to visible light and, in particular, also colorless, so that when introduced inside a packaging for the consumer opening the package, it is as inconspicuous as possible and thus does not or hardly disturb the overall appearance of the packaging.

In preferred embodiments, the indicator substance in the indicator substance dot is a luminescent, in particular fluorescent, dye, where at least one luminescence parameter, such as relative intensity upon excitation, excitation frequency, emission frequency, and/or decay time, depends on the presence of the analyte. In particular, at least one or more of these parameters change continuously with the concentration of the analyte in the fluid to be analyzed, which can in particular be a gas mixture.

The packaging with an indicator label according to the second aspect of the present invention can consist of a planar or bowl-shaped lower part, which is closed on the top side by a film that is at least partially transparent. In this case, the inventive indicator label is preferably adhered to the underside of the top-side film.

In some embodiments of the inventive packaging machine according to the third aspect of the invention, the at least one optical sensor is designed to be manually aligned in a transverse direction relative to a lateral edge of the sealed packaging. Preferably, it can also be done automatically and is therefore, in particular, transversely movable.

Furthermore, the sensor preferably scans indicator labels passing through its detection/scanning range after alignment. This can be achieved by scanning the detection range at a lower scan rate, for example. If such an indicator label is detected during scanning, an optical measurement of the label is performed, for instance by scanning the detection range at a higher measurement rate compared to the scan rate. The scan rate can, for example, be between 50 and 500 Hz, preferably 100 to 200 Hz, and the measurement rate can be between 100 and 2000 Hz, preferably between 500 and 1000 Hz. In further preferred embodiments, the sensor issues a warning or an indication if an expected indicator label is not detected during scanning, for example because it is completely absent or has been partially or fully attached outside the transparent area by the labeling means. The expected presence of the indicator label by the sensor can be determined based on time windows during which the transparent areas of the individual packages pass through the measurement area. These time windows can be pre-calculated based on the known extent of the transparent areas in the longitudinal direction as well as the transport speed of the packaging and a defined starting point. Alternatively, or additionally, the time windows are calculated based on the positions determined by optionally additional optical detection means and the known distances of these detection means along the transport direction of the packaging or the packaging parts monitored by the detection means, such as the formats of the lower packaging parts or the associated sealing films.

Further features, characteristics, and advantages of this invention are apparent from the embodiments presented below with reference to the figures. These are intended solely to illustrate the present invention and in no way to limit its generality.

In detail, it is shown in:

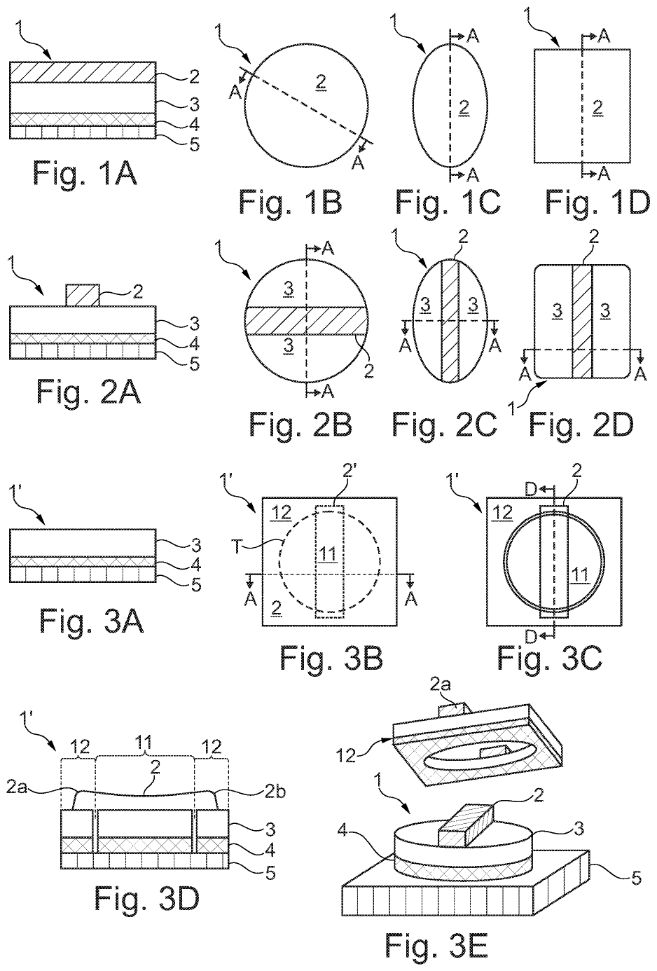

FIG. 1A: A schematic cross-section through an inventive indicator label according to a first embodiment.

FIG. 1B-C: Top views of various variants of the first embodiment of the indicator label.

FIG. 2A: A schematic cross-section through an inventive indicator label according to a second embodiment.

FIG. 2B-C: Top views of various variants of the second embodiment of the indicator label.

FIG. 3A: A cross-section through an indicator label blank representing the starting point of the inventive method for producing an indicator label.

FIG. 3B: A top view of the indicator label blank from FIG. 3A, show-ing the separation line between the label section and the surplus section, as well as the outline of the indicator substance dot to be printed.

FIG. 3C: A top view of the indicator label blank from FIG. 3B after separating the label section and printing the indicator substance dot.

FIG. 3D: A longitudinal cross-section through the indicator label blank from FIG. 3C along the line D-D.

FIG. 3E: A perspective view of a finished indicator label immediately after the surplus section has been removed.

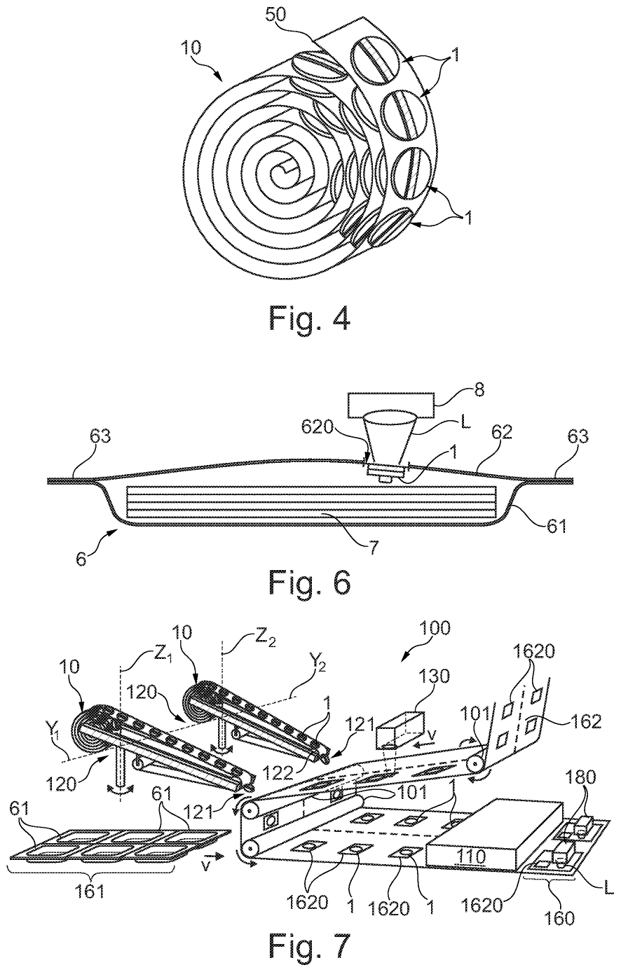

FIG. 4: A schematic representation of an indicator label tape according to the invention in the rolled state with a plurality of indicator labels arranged sequentially on a tape-like peel-off layer.

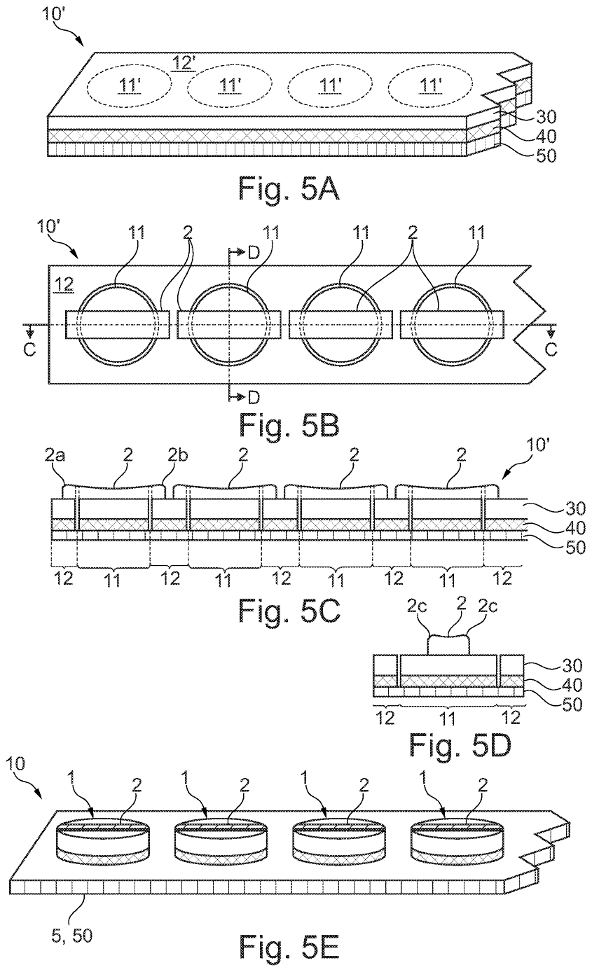

FIG. 5A: A schematic perspective view of the initial section of an indicator label tape blank as the starting point for producing an inventive indicator label tape.

FIG. 5B: A schematic top view of the initial section of the indicator label tape blank from FIG. 5A after dividing the carrier layer into a surplus section and four round label sections, with an indicator substance dot printed on each of the label sections.

FIG. 5C: A schematic longitudinal cross-section through the indicator label tape blank along the line C-C in FIG. 5B.

FIG. 5D: A schematic cross-section along the line D-D in FIG. 5B.

FIG. 5E: A schematic perspective view of the initial section of the finished indicator label tape according to the invention after removing the surplus section of the indicator label tape blank from FIG. 5B.

FIG. 6: A schematic cross-section through an packaging according to the invention with an indicator label according to the invention inside.

FIG. 7: A perspective schematic representation of an packaging machine according to the invention for producing packaging, such as that shown in FIG. 6, using indicator label application means.

FIGS. 1A-1D show various schematic views of several variants of a first embodiment of the inventive indicator label. In FIGS. 1B-1D, three possible variants of the indicator label are shown in a top view. In FIG. 1B the indicator label 1 has a circular outline, in FIG. 1C an oval or an elliptical outline and in FIG. 1D it has a rectangular outline. Other forms of the outline would also be possible, such as a square or a rectangular shape with more or less rounded corners.

All the variants share the cross-section shown in FIG. 1A, which is taken along the dashed lines A-A shown in FIGS. 1B-1D. The indicator label includes a layered structure consisting of a carrier layer 3, which is directly above an adhesive layer 4.

The adhesive layer 4 is used to facilitate adhesion when attaching the indicator label to the inside of a monitored package (see FIG. 6 and the description below). The adhesive layer is terminated by a peel-off or release layer 5 for purely practical reasons before use. Before applying (gluing or attaching) the indicator label, the peel-off layer 5 is removed.

The release layer 5 consists, on the side facing the adhesive layer, of a material to which the adhesive/adhesion promoter of the adhesive layer 4 adheres only weakly, allowing easier removal. The adhesive layer 4 could also be entirely composed of such a weakly adhering material. Examples include fluoropolymer-based plastics such as PTFE. Alternatively, materials such as PTFE-coated paper or cardboard could be used.

The carrier layer 3 serves to mechanically support the indicator substance dot 2, which, in this embodiment, occupies the entire upper side of the indicator label 1 and is therefore also referred to here as the indicator substance layer 2 or simply the indicator layer 2.

The indicator layer 2 contains an indicator substance sensitive to the analyte to be measured in the sense that the presence of the analyte causes an optically detectable change in the properties of the indicator substance. For example, changes may occur in color, structure, absorption, emission properties, or the way the indicator substance reflects polarized light due to binding with or reaction to the analyte.

The indicator substance can also be a luminescent (fluorescent or phosphorescent) dye that emits light at a different, usually lower frequency, in response to excitation by light at a specific frequency. After excitation is turned off, the luminescence response typically decays exponentially with a certain time constant.

For embodiments of the inventive indicator label designed to measure oxygen as the analyte, the indicator substance dot may contain porphyrins as fluorescent dyes. With this class of substances, fluorescence is suppressed with increasing oxygen concentration, so that both the intensity of emitted light at a given excitation decreases and the decay time after turning off the excitation is shortened. By measuring the intensity and/or decay time (after prior calibration), it is possible to determine the oxygen concentration in the atmosphere in contact with the indicator label.

The indicator layer can also contain several different indicator substances. These different substances can be sensitive to various analytes to allow for the detection of the presence and/or concentration of multiple analytes optically. Two or more of the substances in a multi-component indicator layer can also be sensitive to the same analyte but in different ways or with varying intensities to allow redundant measurement or compatibility with different optical sensors.

The advantage of fully covering the upper side of the indicator label with an indicator substance dot in the form of an indicator layer lies in the fact that a larger optical detection area is available compared to if only part of the upper side were covered. This allows for more accurate concentration measurements. Combined with the inventive manufacturing method (described in more detail below), this also ensures that a more uniform thickness of the indicator layer can be achieved.

Specifically, in the manufacturing of an inventive indicator label in accord-ance with the first embodiment of FIGS. 1A-1D, the inventive manufacturing process minimizes deviations in thickness across two spatial dimensions rather than just one. Deviations can thus be kept below a relative variation of 10% or less, or even 1% or less.

FIGS. 2A-2D show various schematic views of variants of the inventive indicator label according to a second preferred embodiment.

The structure, including the layered structure illustrated in FIG. 2A and the possible shapes shown in FIGS. 2B-2D, is similar to that of the first embodiment, with the key difference being that, in the second embodiment, a strip-like indicator substance dot 2 is provided on the upper side of the carrier layer 3 instead of a full-area indicator substance layer as seen in the first embodiment.

Another difference is the (more strongly) rounded corners in the outline of the shape shown in FIG. 2D.

The indicator strip 2 in the second embodiment is flush with the opposite short side ends of the carrier layer 3 but leaves parts of the carrier layer 3 free at the long sides. This results in the total surface area and the amount of indicator substance being significantly smaller than in the first embodiment. This has the advantage of using less of the typically expensive indicator substance per indicator label while still allowing reliable detection and measurement of optical changes in the indicator substance during relative movement between the label 1 and optical sensors.

To ensure this functionality, the indicator strip is designed as long as possible and extends flush with the edge of the carrier layer. When using the label, care must be taken to align the long side of the indicator strip 2 as far as possible parallel to the direction of relative movement.

FIGS. 3A-3E illustrate the manufacturing process according to the invention using the example of producing an indicator label according to the second embodiment.

The process begins with the preparation or production of an indicator label blank 1, shown schematically in FIG. 3A (cross-section) and in FIG. 3B (top view). The blank 1 consists of a layered structure arranged in this order from bottom to top: release layer 5, adhesive layer 4, and carrier layer 3. These layers have properties (composition, thickness, etc.) similar to those used in the first and second embodiments of the indicator label.

The top view in FIG. 3B shows, with dashed lines, the planned segmentation into a circular label section 11 and a surrounding complementary surplus section 12. Overlaid with a dotted line 2′ is the strip-like indicator substance dot 2 to be printed on the upper side of the carrier layer after segmentation. The cross-section shown in FIG. 3A was taken along the cutting line A-A in FIG. 3B.

In FIG. 3C, a top view of the blank 1 is shown after carrying out the two steps of segregating the label section 11 from the surplus section 12 and printing the strip-like indicator substance dot 2 on the carrier layer. As can be seen, the strip-like indicator dot 2 is printed such that its center coincides with the center of the label section 11, and its two opposing short end regions extend into the surplus section 12.

The advantage of this configuration becomes apparent from FIG. 3D, which shows the blank 1′ from FIG. 3C in a longitudinal cross-section along line D-D. It can be observed that the division into the label section 11 and the surplus section 12 runs only through the carrier layer 3 and the adhesive layer 4, while the peel-off layer 5 is not divided. This has the advantage that, even after dividing the label and surplus sections, the indicator strip 2 is kept close to each other for printing purposes.

In alternative embodiments of the method according to the invention, the printing of the indicator strip 2 occurs before the division of the blank 1′ into the two sections, label section 11, and surplus section 12. In these embodiments, it is also possible to cut the peel-off layer 5, thus completely separating the label section 11 from the surplus section. However, the disadvantage is that the finished indicator label may then be difficult to detach from the peel-off layer 5, particularly if the peel-off layer 5 adheres relatively well to the adhesive layer 4 and/or is comparatively thin and flexible. Therefore, even in embodiments of the method where printing occurs first, it is advantageous to divide only the carrier layer 3 and the adhesive layer 4 afterward.

The indicator strip is printed as ink in the form of a liquid suspension, dispersion, or solution of the indicator substance and, if necessary, other substances, such as hydrophobic and/or film-forming substances, onto the top side of the carrier layer 3. The printing process does not play a role in the context of the present invention. The drying of the solvent or solvents in this ink inevitably occurs faster at the edges than in the middle of the strip 2. This leads to the accumulation of indicator substances and other solids at the edges of the strip, forming the thickened areas shown in FIG. 3D at the opposing end regions 2a and 2b of the indicator strip 2. The overhang of the thickened end regions 2a, 2b beyond the edge of the label section 11 is now advantageously chosen so that the part of the indicator strip 2 directly on the label section has a uniform thickness. The remaining thickness variation of the indicator strip above the label section is preferably 10% or less, and even more preferably 1% or less.

From a blank 1 treated in this manner, in a final step illustrated in FIG. 3E, an inventive indicator label 1 can be created by removing, for example by peeling off, the surplus section 12 along with the thickened indicator strip end regions 2a, 2b. To avoid damage to the portion of the indicator strip 2 above the label section during the removal of the surplus section 12 and the thickened ends, preferred embodiments of the method first print the indicator strip and then divide the blank 1′ into the label section 11 and surplus section 12. The division is advantageously performed by punching or cutting, whether mechanically or by laser.

FIG. 4 schematically shows a perspective view of an exemplary embodiment of an inventive indicator label tape, which can, for example, be used in a packaging machine as shown in FIG. 7 and described in detail below. As shown concretely, the tape 10 carries on one side of a strip-like peel-off layer 50, hereinafter also referred to as the peel-off tape 50, a plurality of sequentially arranged indicator labels according to the second embodiment in the variant with a circular outline corresponding to FIGS. 2A, 2B, and 3E. Within the scope of the present invention, however, it is also possible for the peel-off tape 50 to carry other embodiments and/or variants. Different embodiments and/or variants can also coexist on the same tape 10, 50. As shown in the figure, the indicator labels 1 on the peel-off tape 50 are oriented such that the long side of the indicator strip 2 is aligned parallel to the longitudinal direction of the tape 10, 50. However, other orientations are also possible within the context of the invention, for example with the long side of the indicator strip oriented perpendicular to the longitudinal direction of the tape 10, 50.

FIGS. 5A-5E illustrate, in several schematic views, an embodiment of the inventive method for manufacturing the indicator label tape 10 from the preceding FIG. 4.

FIG. 5A shows a perspective view of a front section of the tape blank 10′, with four separable label sections 11′ indicated by dashed lines, which are surrounded by a surplus section 12′. The tape blank 10′ comprises the three stacked, congruent tape-like layers peel-off layer 50, adhesive layer 40, and carrier layer 30.

FIG. 5B shows a schematic plan view of the initial section from FIG. 5A after dividing at least the carrier layer 30, preferably both the carrier layer 30 and the adhesive layer 40, into label sections 11 and a surplus section 12 and printing one indicator strip 2 per label section. The surplus section 12 can also be subdivided into smaller subsections to facilitate later detachment from the peel-off layer. The order of the two steps—division and printing—is arbitrary, i.e., in some manufacturing embodiments, division occurs before printing, while in others, it is the reverse. The first sequence allows the print head for the indicator strips 2 to align precisely with the label sections using the visually detected outline of the division line between 11 and 12. The latter sequence facilitates cleaner detachment, especially ensuring that the indicator strip is not or only slightly damaged.

FIGS. 5C and 5D respectively show a longitudinal section along line C-C and a cross-section along line D-D of FIG. 5B. As indicated in FIG. 5C, after drying, the indicator strips 2 have thickened end regions 2a, 2b. In preferred manufacturing embodiments, the printed strip length 2 is deliberately chosen so that these end regions 2a, 2b lie above the surplus section 12 and are removed together with it in the final step. Then the finished indicator label strip 10 according to the invention, illustrated in FIG. 5E, remains, on whose peel-off strip 50 the indicator labels 1 are lined up as shown. As described above, the division into label sections 11 and excess section(s) 12 can be carried out first, or vice versa. In the first case, each indicator strip 2 forms a continuous block after drying, whereas in the second case the end regions 2a, 2b are separated from the middle region above the label section by a gap during the division/separation, which can be produced, for example, with a punch. These two variants are indicated in FIG. 5C by the dashed lines between the middle regions and the end regions 2a, 2b.

The cross-section in FIG. 5D illustrates the lateral division of the carrier layer 30 and adhesive layer 40 and the thickness profile of the indicator strip 2 in the lateral direction. Similar to the longitudinal direction, the drying of printed indicator substances leads to a profile with thickened edge regions 2c. However, this does not cause a problem during optical measurement as long as the relative motion between the optical sensor and indicator label 1 occurs primarily parallel to the longitudinal direction of the indicator strip 2.

The figures show a band 10 with a row of indicator labels 1 arranged one behind the other. Within the scope of the invention, however, it is equally conceivable to produce a wider band with two, three or more rows of indicator labels. The indicator labels can be arranged on a square, triangular or other grid or even without any specific regular order.

FIG. 6 schematically shows a longitudinal section through a packaging system with an inventive indicator label affixed to its inner side. The packaging 6 consists in a known manner of a bowl-like base 61 covered by a thin film 62 on its upper side and sealed at its edge to ensure a gas-tight closure.

Inside the packaging is the product 7, such as a foodstuff, and a modified atmosphere (not shown), i.e. an atmosphere with a different composition than the rest of the earth's atmosphere, which consists approximately of 79% nitrogen and 21% oxygen plus some trace gases, primarily carbon dioxide. For example, an oxygen-free inert gas atmosphere made of nitrogen and/or noble gases can be present in the packaging to slow down the aging of product 7, i.e. to increase its lifespan or (minimum) shelf life. The aim here would ideally be an oxygen content of 0%. In practice, this is mathematically impossible to achieve anyway, but this goal is missed quite clearly, especially in the case of packaging produced by industrial mass production processes, and residual oxygen contents in the range of 0.5% to 1% are common. However, even such a low-oxygen atmosphere causes a very significant increase in the shelf life of food. The residual oxygen content present in the packaging 6 can now be measured by optically reading the indicator label 1 according to the invention, which is attached to the inside in contact with the atmosphere and has an indicator substance that is sensitive to oxygen. For this purpose, the label is arranged in a transparent area 620 of the upper film 62. Here, “transparent” refers to the light frequencies relevant for the measurement, not necessarily to transparency for visible light.

FIG. 7 schematically illustrates a packaging machine for producing packaging units such as that shown in FIG. 6, for which the machine 100 has, among other things, labeling means for applying indicator labels 1 according to the invention. For example, these are designed here as two arms 120 that can be pivoted about the z and y axes, onto each of which a rolled-up indicator label strip 10 according to the invention is attached, which is unrolled by a deflection roller 122 located in the region of the distal end 121 of the arm 120. Due to the curvature of the strip 10 that occurs during deflection, the label located at the tip 121 comes off at a lower edge. By arranging the deflection rollers 101 which transport the film strip 162 at a speed V in the direction of the arrows, the later inner side of the film 162 is transported to just before the distal end of the labeling arms 120, so that the peeling labels can be glued to the inner side.

The optical detection means 130, for example a camera system, detect, if necessary, transparent areas of the film 162 within which the indicator labels 1 are to be or must be attached in order to be optically detectable from the outside after the packaging has been closed and sealed. The optical detection means 130 are useful when the film 162 has opaque areas for the light used in the optical measurement of the indicator labels, for example because the film is partially printed or vapor-coated with metal or consists of different materials in sections, for example as a combination of a metal and a plastic film, and these areas are not always in the same place, either due to manufacturing tolerances or because films with deliberately different designs are used in one packaging run.

It can then happen that the transparent areas intended and used for the indicator labels, illustrated here as an example as rectangular areas 1620 in two parallel rows in the film 162, are not always located exactly in the same place along the film 162 in the transverse and longitudinal directions. In some embodiments of the packaging machine, longitudinal deviations are compensated for by controlling the unwinding speed of the indicator label strips 10 on the respective arms 120, whereby the speed is increased when the optical detection means report a lower distance between consecutive transparent areas and vice versa, slowed down when the distance is higher. If the labeling arms 120 can also be pivoted about the y-axis y1 or y2 as indicated, longitudinal deviations can also be compensated for alternatively or additionally in this way. Transverse deviations, however, can only be compensated by repositioning the arm tip 121 in the transverse direction. This can be done, as indicated in FIG. 7, by moving it around the respective z-axis z1 or z2. Alternatively (or additionally), the arms 120 can also be mounted so that they can be moved transversely, i.e. in the y-direction. Using other means of the packaging machine 100 (not shown), formats 161 of lower parts 61 for the packaging to be produced are transported at the same speed V. The figure shows a 3×2 arrangement of three packaging lower trays 61 in the longitudinal direction and two in the transverse direction as an example only. Alternative arrangements are also possible. The film 162 is turned downwards by the deflection rollers 101 with the side to which the indicator labels 1 were applied in the transparent areas 1620 by the labeling arms 120 and is sealed onto the formats 161 by the sealing station 110, with a modified atmosphere, such as a nitrogen atmosphere as described above, being introduced into the packaging at the same time. As a result, the indicator labels are located inside the packaging 6 and are in contact with the atmosphere there.

This makes it possible to use the optical sensors 180 to measure the presence or concentration of a gas component of this atmosphere, for example oxygen, or another analyte, such as degradation products such as ammonia or hydrogen sulphide, which indicate advanced aging or spoilage of the packaged goods. A single row of sensors, as shown in FIG. 7, already enables the modified atmosphere created in the packaging 160 to be monitored. If two optical sensors 180 are present one after the other in the transport direction V for each of the lanes—two parallel lanes are shown here, so that n×2 packaging formats can be processed-a leak test can be carried out after sealing, in which the packaging formats 161 are exposed to a rapidly changing external atmospheric pressure immediately after sealing after the atmospheric composition has been determined and a change in the composition of the atmosphere in the packaging is then measured, such as an increase in the oxygen content. Packaging in which a significant change is detected can be discarded after separation (mechanical separation of the individual packages 6 of a format 161) or alternatively, for example if the leak is not very serious, can be given an individual, usually reduced, use-by date.

### LIST OF REFERENCE SYMBOLS

1 Indicator label
1′ Indicator label blank
2 Indicator substance dot/strip/layer
2a, 2b frontal thickening
2c Longitudinal thickening
3 Carrier layer of 1, 1′
4 Adhesive layer of 1, 1′
5 Peel-off layer
10 Indicator label tape
10′ Indicator label tape blank
11 Label section
12 Excess section
30 Tape-shaped carrier layer of 10, 10′
40 Tape-shaped adhesive layer of 10, 10′
50 Peel-off layer of 10, 10′
6 Packaging
61 Bottom part
62 Top film
620 Transparent area in 62
63 Sealing edge, connecting 61 and 62
7 Packaging contents
8 Optical sensor
L Light field
100 Packaging machine
110 Sealing station
120 Attachment arms
121 Distal end of 120
122 Deflection roller
130 Optical detection means
V Speed
y1, y2 y/pitch axis of 120
z1, z2 z/yaw axis of 120
160 Sealed packaging
161 Format consisting of several subsections arranged in a grid pattern
162 Cover film
1620 Transparent areas in 162
180 Optical sensor for detecting and measuring 1

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. A package comprising:
an interior filled with a modified atmosphere; and
an indicator label comprising:
a carrier layer;
an adhesive layer applied flatly on the an underside of the carrier layer; and
an indicator substance dot on an upper side of the carrier layer, which is opposite to the underside, the indicator substance dot comprising an indicator substance that enables a determination of a presence and/or a concentration of an analyte in a multicomponent fluid mixture using an optical sensor through an excitation of the indicator substance with excitation light and reading fluorescence light emitted by the indicator substance using the optical sensor,
wherein:
the indicator substance dot has a uniform thickness in a direction that is perpendicular to the upper side of the carrier layer; and
the indicator label is adhered to a location of the packaging that is sufficiently transparent for excitation and fluorescent light.

15. The package according to claim 14, wherein the packaging encloses a flat or dish-shaped lower part which is closed by a film that is at least partially transparent to excitation and fluorescent light, and the indicator label is adhered in a region of the film that is transparent to excitation and fluorescent light on a side of the film that is in contact with the atmosphere in the packaging.

16. The package according to claim 15, comprising an imprint at the location where the indicator label is adhered, wherein the imprint is sufficiently transparent for excitation and fluorescent light.

17. The package according to claim 16, wherein the imprint comprises an ink that is transparent to the excitation and fluorescent light.

18. The package according to claim 17, wherein an area covered by the indicator label, the imprint includes printed and non-printed areas wherein the non-printed areas make up at least 5% of the area of the indicator label.

19. The package according to claim 17, wherein the imprint comprises a grid of halftone dots applied by digital printing, and a distance between neighboring dots of the grid is 1.05-1.5 times a radius of the halftone dots.

20. A packaging machine for packaging foodstuffs or other sensitive goods in a package that passes through the machine in a transport direction at a transport speed, the packaging machine comprising a labelling device for applying an indicator label to an inner side of the package that is in contact with a modified atmosphere in an interior of the package after the package has been sealed at a specified location where the indicator label can be seen optically from outside the package, the indicator label comprising:
a carrier layer;
an adhesive layer applied flatly on the an underside of the carrier layer; and
an indicator substance dot on an upper side of the carrier layer, which is opposite to the underside, the indicator substance dot comprising an indicator substance that enables a determination of a presence and/or a concentration of an analyte in a multicomponent fluid mixture using an optical sensor through an excitation of the indicator substance with excitation light and reading fluorescence light emitted by the indicator substance using the optical sensor,
wherein:
the indicator substance dot has a uniform thickness in a direction that is perpendicular to the upper side of the carrier layer; and
the specified location is sufficiently transparent for excitation and fluorescent light.

21. The packaging machine according to claim 20, further comprising at least one optical sensor for determining at least one gas component of the atmosphere inside the sealed packages produced by means of the packaging machine, wherein the optical sensor is aligned with the specified location, which is based on a predefined reference point or reference line of an individual package, of a format of a plurality of packages or of a top-side cover foil.

22. The packaging machine according to claim 21, wherein the optical sensor is prepared:
to be manually or automatically aligned in a transverse direction relative to a lateral edge of the sealed packages;
after alignment, to scan for indicator labels travelling through a field of view of the sensor;
to optically measure recognized indicator labels to determine the gas component; and
to report the absence or insufficient legibility of an expected indicator label.

23. The packaging machine according to claim 21, further comprising an optical detector for dynamically identifying transparent areas of individual unsealed packages or packaging components and determining their position, wherein:
the labelling device is prepared to apply the indicator labels within transparent area detected by the optical detector; and/or
the optical sensor is mounted on a transverse carriage and configured to automatically align itself with the determined position of the detected transparent area using the carriage.

24. A method for packaging foodstuffs or other sensitive goods in packages according claim 14 and post-controlling a quality of a modified atmosphere in the packaging by optical measurement of at least one gas component of the modified atmosphere, comprising:
for a package which has not yet been sealed, applying an indicator label according to one of the above indicator label claims using a labelling device to the inside of an arbitrarily predetermined sufficiently transparent area which is in contact with the atmosphere inside the package after it has been sealed;
using an optical sensor manually or automatically aligned with a specified area:
scanning for indicator labels travelling through a detection area of the optical sensor; and
optically measuring recognized indicator labels, wherein, when the optical sensor does not detect an indicator label within a known time window corresponding to a crossing of the sufficiently transparent area of one of the packages through the detection area of the optical sensor, outputting a warning signal and physically or virtually marking the affected package.
